(19) United States
(12) Patent Application Publication
(10) Pub. No.: US 2025/0061570 A1
(43) Pub. Date: Feb. 20, 2025

(54) METHOD AND APPARATUS FOR POST-PROCESSING RADIOGRAPHS

(71) Applicant: Siemens Healthineers AG, Forchheim (DE)

(72) Inventors: Ludwig RITSCHL, Buttenheim (DE); Magdalena HERBST, Erlangen (DE); Marcel BEISTER, Erlangen (DE); Jamin SCHAEFER, Erlangen (DE)

(73) Assignee: Siemens Healthineers AG, Forchheim (DE)

(21) Appl. No.: 18/800,591

(22) Filed: Aug. 12, 2024

(30) Foreign Application Priority Data
Aug. 14, 2023 (EP) 23191290.8

(51) Int. Cl. G06T 7/00; G06T 7/11

(52) U.S. Cl. CPC G06T 7/0012; G06T 7/11; G06T 2207/10036; G06T 2207/30008; G06T 2207/30024

(57) ABSTRACT

A method for post-processing radiographs from radiographic data including spectral recordings of a region of interest of an object, the method comprising: creating, from the radiographs, a first image in which a first material of the object is highlighted and a second material of the object is suppressed; creating, from the radiographs, a second image in which the second material is highlighted and the first material is suppressed; post-processing the images, wherein the first image is post-processed by a first filter module and the second image is post-processed by a second filter module; combining the post-processed images to form a single image depicting the first material and the second material; and outputting the single image.

### CROSS-REFERENCE TO RELATED APPLICATION(S)

The present application claims priority under 35 U.S.C. § 119 to European Patent Application No. 23191290.8, filed Aug. 14, 2023, the entire contents of which is incorporated herein by reference.

### FIELD

One or more example embodiments of the present invention relate to a method and an apparatus for post-processing radiographs and a control facility (or device) for a radiography system and a radiography system with such a control facility (or device).

### BACKGROUND

Radiography is a widely used method in medicine for producing recordings of the interior of the body. An X-ray beam is radiated through the body onto a detector (formerly a film) resulting in a radiograph or an X-ray image depicting a projection of the body onto a plane. In addition to native recordings, it is also possible to take recordings after the administration of a contrast agent, often iodine, for example for angiography.

To enable visual interpretation of radiographs, a plurality of image processing steps are typically performed. An image processing pipeline can be roughly divided into physically motivated corrections (for example, log(I/10) or scattered beam correction), dynamic range compression (DRC) and high-frequency feature enhancement (HFE).

The main motivation for the DRC step consists in eliminating the low-frequency brightness modulation of an image caused by the varying projected thickness of the object. Herein, the relative bone contrasts within this object must be retained. A standard method used for this is to apply a low-pass filter (Gaussian or bilateral) and perform a non-linear transformation of this image that reduces its dynamic range, for example by applying an S-shaped transformation function. After this transformation, any remaining high frequencies are added back.

If the low-pass filter cannot completely separate the bone from the soft-tissue structure, the DRC automatically removes low-frequency bone contrast in regions with lower object thickness, which is a serious problem. Extremity images in particular pose a challenge here, since the background that needs to be compensated can vary greatly, for example, an image of a foot including the toes.

The main motivation for the HFE step is to improve diagnostically relevant structures compared to anatomical noise and quantum background noise.

Both filtering methods entail the problem that, on transition from high-contrast regions to low-contrast regions, a systematic error can occur during automated filtering. To date, there has been no solution to this problem. The only remedy is to reduce the strength of DRC or HFE, which often leads to a typically “analog” or “film-like” image impression.

### SUMMARY

It is an object of one or more embodiments of the present invention to disclose an alternative, more convenient method and a corresponding apparatus for post-processing radiographs with which the above-described disadvantages are avoided.

At least this object is achieved by a method, an apparatus, a control facility (or device) and/or a radiography system according to one or more embodiments of the present invention.

A method according to the method is used for the post-processing of radiographs from radiographic data comprising spectral recordings of a region of interest of an object, for example, a body region of a patient. An X-ray system capable of recording such spectral data is in particular an X-ray system with photon-counting detectors, an X-ray system with a dual-layer detector or multi-layer detector, an X-ray system which records two consecutive images with different beam energies (kV) and/or filters.

The method comprises the following steps:
creating a first image from the radiographs in which a first material of the object, which has different spectral attenuation properties than a second material of the object, is highlighted and the second material is suppressed,
creating a second image from the radiographs in which the second material is highlighted and the first material is suppressed,
post-processing the images created via filter modules, wherein the first image is post-processed by a first filter module and the second image is post-processed by a second filter module,
combining the post-processed images to form a single image in which the first material and the second material are depicted,
outputting the single image.

The creation of an image is in principle known to the person skilled in the art. In the case described here, radiographs are provided as raw data (for example, projection images) and material decomposition takes place based on these radiographs. For purposes of simplicity, here it can, for example, be assumed that an object consists of only two materials. The (material-decomposing) creation then provides two images.

Spectral recordings of a region of interest often have two or more raw images that represent the region of interest at different beam energies in each case. There may also be a dataset having a vector of two or more image values at each pixel position. On the creation of an image, the image value of each pixel of the image created is typically calculated from a link based on corresponding pixels of the raw images (or vector entries). However, a pixel group of the raw images around the corresponding pixel can also be considered for each pixel of the resulting image. Preferably, the calculation comprises a weighted subtraction in which, for example, the value X of a corresponding pixel of the image created with the coefficients a and b is calculated from the values A and B of corresponding pixels of two raw images using the formula X=aA−bB. Herein, the coefficients preferably represent weightings. In addition to subtraction, a non-linear combination of the input values can also be calculated. If desired, a lookup table can also be used instead of an analytical function.

Herein, (at least) two images are created that depict the two materials of the recorded object differently. These two materials differ in that the first material has different spectral attenuation properties (for example, absorbs more radiation) than the second material. Herein, preferably, the first material is bone material or a contrast agent (for example, iodine) and the second material is tissue.

In the first image, the first material (for example, bone) is highlighted and the second material (for example, surrounding tissue) is suppressed; in the second image, the exact opposite is the case: the second material (for example, tissue) is highlighted and the first material (for example, bone) is suppressed. In the example of bone or tissue, the first image could be called a “bone image” and the second image could be called a VNBC image (VNBC: “virtual non-bone contrast”). Here, it should be noted that, in contrast to “virtual non-calcium” images (VNCa images), with respect to embodiments of the present invention, a decomposition is preferably shown in which a local calcium contrast is replaced by water and not set to 0. Setting to “0” (as is often done in the prior art) would often show a negative contrast in the projection image, which could be disadvantageous here.

Since images are being discussed here, it is obvious that “highlighting” or “suppressing” a material, corresponds to “highlighting” or “suppressing” image information for this material. For example, pixel values of an image can be reduced to suppress a material or increased to highlight a material. With inverse images, exactly the opposite can be the case.

The images created (first image and second image and possibly further images) are then post-processed by filter modules. These filter modules preferably comprise the aforementioned DRC filters and HFE filters, i.e., a compressor and a filter for high-frequency feature enhancement. As stated in the introduction, the application of these filters to an image is known from the prior art. Herein, the difference from the prior art is that the two images are created by (physical) material decomposition (and not by image-based decomposition) and the first image is post-processed by a first filter module and the second image by a second filter module. Thus, these filter modules can be optimized for the type of images, i.e., they can differ. For example, the first filter module can comprise a DRC filter and an HFE filter that have been optimized for a bone image and the second filter module can comprise a DRC filter and an HFE filter that have been optimized for a VNBC image. In addition, further images can also be post-processed by further filter modules, wherein each filter module should be used for an image (preferably for an image type) for which it has been optimized.

After each image has been post-processed, and has thus in particular undergone optimum filtering, the post-processed images are combined to form a single image. Post-processed images are at least the first image and the second image after post-processing. Further post-processed images can be added, but they can also be combined to form a further single image. Since the first material (for example, bone) is highlighted in the first image and the second material (for example, tissue) is highlighted in the second image, the single image shows both the first material and the second material (i.e., for example bone with surrounding tissue). The single image is then output, for example, for assessment by a person.

An apparatus according to embodiments of the present invention is used for post-processing radiographs from radiographic data comprising spectral recordings of a region of interest of an object. It is preferably designed to execute the method, according to embodiments of the present invention, and comprises the following components:
an image unit designed to create a first image from the radiographs in which a first material of the object, which has different spectral attenuation properties than a second material of the object is highlighted and the second material is suppressed, and to create a second image from the radiographs in which the second material is highlighted and the first material is suppressed,
a post-processing unit designed to post-process the images created by filter modules, wherein the first image is post-processed by a first filter module and the second image is post-processed by a second filter module,
a combining unit designed to combine the post-processed images to form a single image in which the first material and the second material are depicted,
an output unit designed to output the single image.

The function of the components has already been described above in the context of the method. The apparatus is particularly preferably (specially) designed to perform the method according to embodiments of the present invention.

A control facility (also referred to herein as a control device), according to embodiments of the present invention, for controlling a radiography system comprises an apparatus, according to embodiments of the present invention, and/or is designed to execute a method according to embodiments of the present invention.

A radiography system according to embodiments of the present invention comprises a control facility according to embodiments of the present invention.

A large part of the aforementioned components of the apparatus can be implemented in whole or in part in the form of software modules in a processor of a corresponding computer system, for example by a control facility of a radiography system. An extensively software-based implementation has the advantage that it is also possible to retrofit computer systems used to date in a simple way by a software update in order to work in the manner according to embodiments of the present invention. Insofar, the object is also achieved by a corresponding non-transitory computer program product with a computer program, which can be loaded directly into a computer system, with program segments for executing the steps of the method according to embodiments of the present invention, or at least the steps that can be executed by a computer, when the program is executed in the computer system. In addition to the computer program, such a computer program product can optionally comprise additional parts, such as, for example, documentation and/or additional components, including hardware components, such as, for example, hardware keys (dongles etc.) for using the software.

Transportation to the computer system or to the control facility and/or for storage on or in the computer system or the control facility can take place via a computer-readable medium, for example a memory stick, a hard disk or another kind of transportable or permanently installed data carrier on which the program segments of the computer program that can be read-in and executed by a computer system are stored. For this purpose, the computer system can, for example, have one or more interacting microprocessors or the like.

Further, particularly advantageous embodiments and developments of the present invention emerge from the dependent claims and the following description, wherein the claims of one claim category can also be developed analogously to the claims and descriptive parts to form another claim category and, in particular, individual features of different exemplary embodiments or variants can also be combined to form new exemplary embodiments or variants.

According to a preferred method, the first material is bone and/or a contrast agent, in particular iodine. The second material is preferably (surrounding) tissue. The first image is preferably constructed in such a way that bones of a patient and/or the contrast agent are highlighted and information relating to the surrounding tissue of the patient is suppressed. The second image is preferably constructed in such a way that the tissue of the patient is highlighted and information relating to bones of the patient and/or the contrast agent is suppressed.

As already mentioned, when a material is highlighted, image values of pixels that show this material are increased (or reduced in an inverted image). When a material is suppressed, image values of pixels that show this material are reduced (or increased in an inverted image).

According to a preferred method, in a spectral recording of a region of interest (for example, a region of a patient's body), a plurality of raw images are available or created as radiographs. The first image and the second image are then preferably created by a number of weighted subtractions of two raw images, wherein in each case a weighting factor defines which material is highlighted in these images and which material is suppressed. This can, for example, be achieved by the aforementioned function X=aA−bB for the individual image points, wherein a and b are the weighting factors. Herein, the two images are created from the same raw images.

According to a preferred method, the second image shows a decomposition of raw images in which a local contrast, in particular a calcium contrast and/or a contrast agent contrast, in particular an iodine contrast has been virtually replaced by water. As mentioned above, this has the advantage that negative contrast is prevented.

According to a preferred method, the at least two different beam energies of a spectral recording result from acceleration voltages that have a difference of at least 30 kV, in particular at least 50 kV. Alternatively or additionally, (for example, when irradiating with a broad X-ray spectrum), the intermediate beam energies preferably have a difference of at least 30 keV, in particular at least 50 keV.

According to a preferred method, a filter module comprises a compressor (a DRC filter) which compresses the dynamic range of the image. A compressor removes the low-frequency brightness modulation of an image caused by the varying projected thickness of the object. A standard method for this is to apply a low-pass filter (Gaussian or bilateral) and perform a non-linear transformation of this image that reduces its dynamic range, for example by applying an S-shaped transformation function. After this transformation, any remaining high frequencies are added back.

According to a preferred method, a filter module comprises a feature enhancement filter (“HFE filter”: “high-frequency feature enhancement filter”). An HFE filter improves diagnostically relevant structures compared to anatomical noise and quantum background noise. A serial application of a compressor (DRC filter) and a feature enhancement filter (HFE filter) is also preferred.

According to a preferred method, a filter module comprises a denoising filter. Such filters are known in the prior art.

According to a preferred method, the first filter module differs from the second filter module. The filter modules should be optimized for the images to which they are applied. For example, the first filter module for the first image should be optimized for first structures, i.e., for structures, that absorb relatively strongly. The second filter module for the second image should be optimized for second structures, i.e., for structures which absorb relatively weakly. Herein, it is preferable for the filter modules to have the same filter types and/or the same filter architecture, while parameters of the filters are different. In a preferred example, both filters have a DRC filter and a HFE filter (and possibly also a denoising filter), which are in particular also applied in the same order.

The use of AI-based methods (AI: “artificial intelligence”) is preferred for the method according to embodiments of the present invention. Artificial intelligence is based on the principle of machine-based learning and is usually performed with a learning algorithm that has been trained accordingly. Machine-based learning is often referred to as “machine learning”, which also includes the principle of “deep learning”. For example, a deep convolutional neural network (DCNN) is trained to create the first and the second image from two radiographs as raw images. However, the filters of a filter module or even a filter module can be implemented by AI.

Preferably, components of embodiments of the present invention, in particular the image unit and/or the post-processing unit are provided as a “cloud service”. Such a cloud service is used to process data, in particular via artificial intelligence, but can also be a service based on conventional algorithms or a service in which an evaluation by humans takes place in the background. In general, a cloud service (hereinafter also “cloud” for short) is an IT infrastructure in which, for example, storage space or computing power and/or application software is provided via a network. Herein, communication between the user and the cloud takes place via data interfaces and/or data transmission protocols. In the present case, it is particularly preferable for the cloud service to provide both computing power and application software.

In the context of a preferred method, data is provided to the cloud service via the network. This comprises a computer system, for example a computer cluster, which generally does not comprise the user's local computer. This cloud can in particular be provided by the medical facility that also comprises the medical systems. For example, the data for image recording is sent to a (remote) computer system (the cloud) via a RIS (radiology information system) or PACS. Preferably, the cloud computer system, the network and the radiography system represent a network in terms of data technology. Herein, the method can be implemented via a constellation of instructions in the network. The data calculated in the cloud (“result data”) is later returned to the user's local computer via the network.

### BRIEF DESCRIPTION OF THE DRAWINGS

The present invention is explained in more detail below with reference to the attached figures and with reference to exemplary embodiments. Herein, the same components are given identical reference symbols in the various figures. The figures are generally not to scale. The figures show:

FIG. 1 an example of a radiography system with a control facility according to embodiments of the present invention,

FIG. 2 a sketch of image post-processing according to the prior art,

FIG. 3 a sketch of image post-processing according to the method according to embodiments of the present invention.

### DETAILED DESCRIPTION

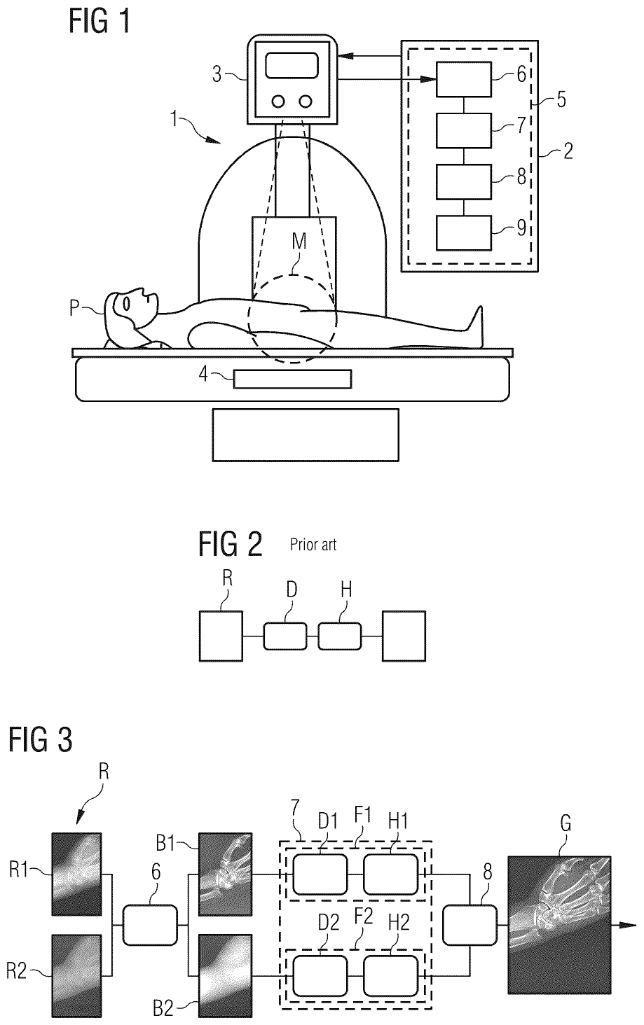

FIG. 1 is a rough schematic view of a radiography system 1 with a control facility 2. This is equipped with an apparatus 5 designed to perform the method according to embodiments of the present invention. As usual, the radiography system 1 has a radiation source 3, here an X-ray source, and irradiates a patient P during radiographic recording so that the radiation hits a detector 4 located opposite the radiation source 3 in each case. Herein, the radiography system 1 is designed to produce spectral recordings and is, for example, a system that can generate two different acceleration voltages for the radiation or has a detector 4 that can measure with energy resolution. The radiography system 1 can also comprise selective filters that can be used to filter two different beam energies.

With respect to the control facility 2, only components essential for the explanation of the present invention are depicted. In principle, radiography systems 1 and associated control facilities 2 are known to the person skilled in the art and therefore do not need to be explained in detail.

The control facility 2 comprises an apparatus 5 for post-processing radiographs R from radiographic data comprising spectral radiographs of a region of interest M of an object P, for example, as depicted here, a body region M of a patient P, at two different beam energies.

The apparatus comprises an image unit 6, a post-processing unit 7, a combining unit 8 and an output unit 9, which can be implemented by a simple data interface. For a description of the function of the components, see also FIG. 3.

The image unit 6 is designed to create at least two different images B1, B2 from the radiographs R. Herein, the first image B1 shows the bones of the patient P as prominent and the tissue is suppressed. Conversely, the second image B2 shows the tissue and the bones to a lesser extent. However, instead of the bones, it is also possible to take account of a contrast agent, for example iodine.

The post-processing unit 7 is designed to post-process the images created B1, B2 by filter modules F1, F2, wherein the first image B1 is post-processed by a first filter module F1 and the second image B2 is post-processed by a second filter module F2. It is quite possible for these filter modules F1, F2 to differ and this constitutes a particular advantage of one or more embodiments of the present invention. Namely, each of the images B1, B2 can be post-processed by filter modules F1, F2 optimized for these images B1, B2. Herein, it is preferable for the filter modules F1, F2 to have the same types of filters D1, D2, H1, H2 and/or the same architecture of filters D1, D2, H1, H2, while the parameters of the filters D1, D2, H1, H2 are different.

The combining unit 8 is designed to combine the post-processed images B1, B2 to form a single image G. This single image depicts the first material and the second material.

The output unit 9 is used to output the single image G, for example for diagnosis.

FIG. 2 shows a sketch of image post-processing according to the prior art. In the example shown, a radiograph R (all recorded images) is post-processed by the same filters D, H.

FIG. 3 is a sketch of an example of the method, according to embodiments of the present invention, for post-processing spectral radiographs R from radiographic data that has, for example, been recorded with a radiography system 1 according to FIG. 1 at two different beam energies. The radiographs R produced were two raw images R1, R2, which are depicted on the far left. The beam energies should be quite far apart and not overlap so that different information is available in the raw images R1, R2.

The image unit 6 is now used to create a first image B1 and a second image B2 from the raw images R1, R2 and herein specific materials are suppressed or highlighted. In this example, the first material is bones and the second material is surrounding tissue. Herein, the first image B1 is constructed in such a way that the bones are highlighted and information relating to the surrounding tissue of the patient P is suppressed, and the second image B2 is constructed in such a way that the tissue is highlighted and information relating to bones is suppressed.

Then, the images created B1, B2 are suppressed by filter modules F1, F2 of the post-processing unit 7. In this example, each filter module F1, F2 comprises a compressor D1, D2 (or a DRC filter D1, D2), which compresses the dynamic range of the image, and a feature enhancement filter H1, H2 (or an HFE filter H1, H2). Herein, the first image B1 is post-processed by the first filter module F1 and the second image B2 with the second filter module F2.

After this post-processing, the post-processed images B1, B2 are combined by the combining unit 8 to form a single image G and output.

Finally, reference is made once again to the fact that the figures described in detail above are exemplary embodiments only and can be modified by the person skilled in the art in a wide variety of ways without departing from the scope of the present invention. Furthermore, the use of the indefinite article “a” or “an” does not preclude the possibility that the features may also be present on a multiple basis. Likewise, the terms “unit” and “device” do not preclude the possibility that the components in question could consist of a plurality of interacting sub-components which could possibly also be spatially distributed. The expression “a number” should be understood as meaning “at least one”.

Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the present invention has been shown and described with respect to certain example embodiments, equivalents and modifications will occur to others skilled in the art upon the reading and understanding of the specification. The present invention includes all such equivalents and modifications and is limited only by the scope of the appended claims.

What is claimed is:

1. A method for post-processing radiographs from radiographic data including spectral recordings of a region of interest of an object, the method comprising:
creating, from the radiographs, a first image in which a first material of the object is highlighted and a second material of the object is suppressed, the first material having different spectral attenuation properties than the second material;
creating, from the radiographs, a second image in which the second material is highlighted and the first material is suppressed;
post-processing the first image and the second image, wherein the first image is post-processed by a first filter module and the second image is post-processed by a second filter module;
combining the post-processed first image and the post-processed second image to form a single image in which the first material and the second material are depicted; and
outputting the single image.

2. The method as claimed in claim 1, wherein
the first material is at least one of bones or a contrast agent,
the second material is surrounding tissue,
the first image is constructed such that the at least one of the bones or the contrast agent are highlighted and such that information relating to the surrounding tissue is suppressed,
the second image is constructed such that the surrounding tissue is highlighted and such that information relating to the at least one of the bones or the contrast agent is suppressed.

3. The method as claimed in claim 1, wherein, in a spectral recording of the region of interest, a plurality of raw images are available or created as the radiographs and the first image and the second image are calculated by a number of weighted subtractions of two of the plurality of raw images, wherein in each case a weighting factor defines which material is highlighted and which material is suppressed.

4. The method as claimed in claim 1, wherein the second image shows a decomposition of raw images in which a local contrast has been virtually replaced by water, wherein the local contrast includes at least one of a calcium contrast or a contrast agent contrast.

5. The method as claimed in claim 1, wherein the radiographs are recorded with an X-ray system that at least one of includes photon-counting detectors,
includes a dual-layer detector,
includes a multi-layer detector,
records two consecutive images with different beam energies, or
records two consecutive images with different filters.

6. The method as claimed in claim 1, wherein
at least one of the first filter module or the second filter module includes at least one of
a compressor configured to compress a dynamic range of an image,
a feature enhancement filter, or
a denoising filter, and
the compressor and the feature enhancement filter are in series.

7. The method as claimed in claim 1, wherein
the first filter module has different parameters from the second filter module,
the first filter module is a same type of filter as the second filter module, and
the first filter module and the second filter module have a same architecture.

8. An apparatus for post-processing radiographs from radiographic data including spectral recordings of a region of interest of an object, the apparatus comprising:
an image unit configured to
create, from the radiographs, a first image in which a first material of the object is highlighted and a second material of the object is suppressed, the first material having different spectral attenuation properties than the second material, and
create, from the radiographs, a second image in which the second material is highlighted and the first material is suppressed;
a post-processing unit configured to post-process the first image and the second image, wherein the first image is post-processed by a first filter module and the second image is post-processed by a second filter module;
a combining unit configured to combine the post-processed first image and the post-processed second image to form a single image in which the first material and the second material are depicted; and
an output unit configured to output the single image.

9. A control device configured to execute the method as claimed in claim 1.

10. A radiography system comprising the control device as claimed in claim 9.

11. A non-transitory computer program product including instructions that, when executed by a computer, cause the computer to execute the method as claimed in claim 1.

12. A non-transitory computer-readable storage medium storing instructions that, when executed by a computer, cause the computer to perform the method of claim 1.

13. The method as claimed in claim 1, wherein the second image shows a decomposition of raw images in which a local contrast has been virtually replaced by water.

14. The method as claimed in claim 1, wherein
at least one of the first filter module or the second filter module includes at least one of
a compressor configured to compress a dynamic range of an image,
a feature enhancement filter, or
a denoising filter.

15. The method as claimed in claim 1, wherein the first filter module differs from the second filter module.

16. The method as claimed in claim 2, wherein, in a spectral recording of the region of interest, a plurality of raw images are available or created as the radiographs and the first image and the second image are calculated by a number of weighted subtractions of two of the plurality of raw images, wherein in each case a weighting factor defines which material is highlighted and which material is suppressed.

17. The method as claimed in claim 16, wherein
at least one of the first filter module or the second filter module includes at least one of
a compressor configured to compress a dynamic range of an image,
a feature enhancement filter, or
a denoising filter, and
the compressor and the feature enhancement filter are in series.

18. The method as claimed in claim 16, wherein
the first filter module has different parameters from the second filter module,
the first filter module is a same type of filter as the second filter module, and
the first filter module and the second filter module have a same architecture.

19. The method as claimed in claim 2, wherein
at least one of the first filter module or the second filter module includes at least one of
a compressor configured to compress a range of the image,
a feature enhancement filter, or
a denoising filter, and
the compressor and the feature enhancement filter are in series.

20. The method as claimed in claim 2, wherein
the first filter module has different parameters from the second filter module,
the first filter module is a same type of filter as the second filter module, and
the first filter module and the second filter module have a same architecture.
